(19) United States
(12) Patent Application Publication
(10) Pub. No.: US 2001/0053852 A1
(43) Pub. Date: Dec. 20, 2001

(54) Process for preparing alkyl- or aryloxyacetaldehydes

(76) Inventors: Karlheinz Giselbrecht, Pasching (AT); Rudolf Hermanseder, Pennewang (AT)

Correspondence Address: WENDEROTH, LIND & PONACK, L.L.P., 2033 K STREET N. W., SUITE 800, WASHINGTON, DC, 20006-1021, US

(21) Appl. No.: 09/897,390

(22) Filed: Jul. 3, 2001

(30) Foreign Application Priority Data
May 7, 2000 (AT) A 1153/2000

(51) Int. Cl. C07D 237/02; C07C 45/42

(52) U.S. Cl. 544/224; 568/426; 568/448

(57) ABSTRACT

A process for preparing alkyl- or aryloxyacetaldehydes of the formula

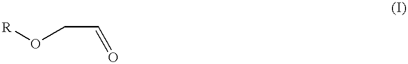

where R can be an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle, which comprises reacting a compound of the formula

R—OM (II)

where R is as defined above and M can be an alkali metal atom or an alkaline earth metal atom, with a compound of the formula

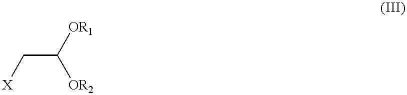

where R₁ and R₂ independently of one another are a C₁-C₆-alkyl radical or together are a C₂-C₆-alkylene radical and X is a halogen atom, to form the corresponding dialkylacetal of the formula

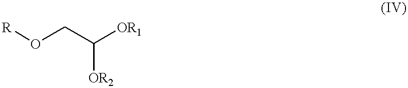

where R, R₁ and R₂ are as defined above, whereupon acetal cleavage is carried out to give the desired alkyl- or aryloxyacetaldehydes of the formula (I).

The invention relates to a process for preparing alkyl- or aryloxyacetaldehydes from the corresponding alkoxides via the diacetals with subsequent acetal cleavage.

Alkyl- or aryloxyacetaldehydes are valuable starting products in organic synthesis. Thus, they are used, for example, as starting material for preparing pharmaceutical and agricultural chemicals.

To prepare alkyl- or aryloxyacetaldehydes, for instance benzyloxyacetaldehyde, a number of variant methods are already described in the literature. One potential method, is, for example, the metaperiodate cleavage of diols, for example glycerol, described in J. Org. Chem. (1997), 62(8), 2622-2624. An alternative is the NaIO₄ cleavage of substituted glycerol acetonides described in Synth. Commun. (1988), 18(4), 359-66.

The desired acyloxyacetaldehydes can, however, also be prepared by a Swern oxidation starting from, for example, 2-(benzyloxy)ethanol in accordance with J. Org. Chem. (1988), 53(18), 4274-82).

The disadvantages of the previously known preparation variants are, inter alia, due to the use of critical oxidizing agents, such as periodate etc., and/or due to the starting materials which are not readily accessible or are expensive.

The object of the invention was to find a novel process for preparing alkyl- or aryloxyacetaldehydes which starts from readily accessible starting materials and leads to the desired end product in a few simple steps.

Unexpectedly, this object was achieved by using haloacetaldehyde dialkylacetals and alkoxides as starting compounds.

The invention therefore relates to a process for preparing alkyl- or aryloxyacetaldehydes of the formula

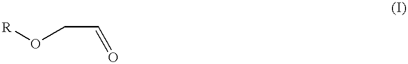

where R can be an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle, which comprises reacting a compound of the formula

R—OM (II)

where R is as defined above and M can be an alkali metal atom or an alkaline earth metal atom, with a compound of the formula

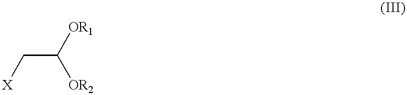

where R₁ and R₂ independently of one another are a C₁-C₆-alkyl radical or together are a C₂-C₆-alkylene radical and X is a halogen atom, to form the corresponding dialkylacetal of the formula

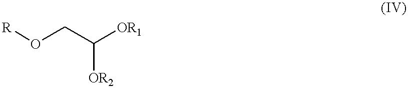

where R, R₁ and R₂ are as defined above, whereupon acetal cleavage is carried out to give the desired alkyl- or aryloxyacetaldehyde of the formula (I).

In the inventive process, alkyl- or aryloxyacetaldehydes of the formula (I) are prepared. In the formula (I) R is an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle.

Alkyl here is taken to mean saturated or mono- or polyunsaturated, unbranched, branched or cyclic primary, secondary or tertiary hydrocarbon radicals. These are, for example, C₁-C₂₀-alkyl radicals, for example methyl, ethyl, propyl, isopropyl, butyl, isobutyl, t-butyl, pentyl, cyclopentyl, isopentyl, neopentyl, hexyl, isohexyl, cyclohexyl, cyclohexyl-methyl, 3-methylpentyl, 2,2-dimethylbutyl, 2,3-di-methylbutyl, octyl, cyclooctyl, decyl, cyclodecyl, dodecyl, cyclododecyl etc. Preference is given here to C₁-C₁₂-alkyl radicals, and particular preference to C₂-C₈-alkyl radicals. The alkyl group may be unsubstituted or monosubstituted or polysubstituted by substituents which are inert under the reaction conditions. Suitable substituents are, for example, carboxylic esters or amides, alkoxy, preferably C₁-C₆-alkoxy, aryloxy, preferably C₆-C₂₀-aryloxy, nitro, cyano, sulfonic esters or amides etc.

Aryl is preferably C₆-C₂O-aryl groups, for example phenyl, biphenyl, naphthyl, indenyl, fluorenyl etc.

The aryl group here may be unsubstituted or mono- or polysubstituted by substituents which are inert under the reaction conditions. Suitable substituents in this case are again carboxylic esters or amides, alkoxy, preferably C₁-C₆-alkoxy, aryloxy, preferably C₆-C₂₀-aryloxy, nitro, cycano, sulfonic esters or amides etc.

Alkaryl or alkylaryl are alkyl groups which have an aryl substituent, for instance benzyl. Aralkyl or arylalkyl relates to an aryl group having an alkyl substituent.

Heteroaryl or heterocycle are cyclic radicals which contain at least one O or N atom in the ring. These are, for example, furyl, pyridyl, pyrimidyl, imidazolyl, tetrazolyl, pyrazinyl, benzofuranyl, quinolyl, isoquinolyl, isobenzofuryl, pyrazolyl, indolyl, isoindolyl, benzoimidazolyl, purinyl, carbazolyl, oxazolyl, isoxazolyl, pyrrolyl, quinazolinyl, pyridazinyl, phthalazinyl etc.

Functional O or N groups can if necessary be protected here. The heteroaryl group or the heterocycle can be unsubstituted or mono- or polysubstituted by the substituents already listed above.

Alkylheteroalkyl or alkylheterocycle are alkyl groups which are substituted by a heteroaryl group or by a heterocycle, respectively.

Preferred compounds of the formula (I) are those where R is an unsubstituted or mono- or polysubstituted C₁-C₁₂-alkyl radical, particularly preferably a C₂-C₈-alkyl radical or an alkylaryl radical having 1-12 carbon atoms in the alkyl moiety, particularly preferably benzyl.

Preferred substituents are carboxylic esters or carboxylic amides, C₁-C₆-alkoxy, C₆-C₂₀-aryloxy, nitro or cyano.

Particularly preferably, the radical R is unsubstituted.

To prepare the compounds of the formula (I), according to the invention a compound of the formula (II) is reacted with a compound of the formula (III). In the formula (II), R is as defined in the formula (I) and M is an alkali metal or an alkaline earth metal atom. Preferred alkali metal atoms or alkaline earth metal atoms are Li, Na, K, Ca, Mg, Cs. Particular preference is given to Na or K.

The compounds of the formula (II) are generally commercially available in large amounts and inexpensively, or they can be prepared in a simple manner, for example by reacting the corresponding alcohol ROH with an alkoxide MOalkyl, for example with sodium methoxide, in an alcohol alkylOH, for example methanol, as solvent.

In the formula (III), R₁ and R₂ independently of one another are a C₁-C₆-alkyl radical, preferably a C₁-C₄-alkyl radical.

The alkyl radical can be saturated, unbranched, branched or cyclic. Preference is given to unbranched or branched alkyl radicals, such as methyl, ethyl, propyl, isopropyl, butyl, hexyl. Particular preference is given to methyl, ethyl and propyl. R₁ and R₂, however, can also together be a C₂-C₆-alkenyl radical, so that a cyclic acetal is formed. C₂-C₆-alkenyl radicals are ethylene, propylene, butylene, pentylene and hexylene in this case. Preference is given to C₂-C₄-alkylene radicals.

X in formula (III) is halogen.

Preferably, X is Cl or Br; particularly preferably Cl.

The compounds of the formula (II) and the formula (III) are used according to the invention in an equimolar amount or one of the two compounds is used in a molar excess, preferably the compound of the formula (II) being used in a molar excess of from 1.1 to 2 mol per mole of compound of the formula (III).

The reaction may be carried out in an organic solvent. Suitable solvents are those which are inert under the reaction conditions. Preferably, higher-boiling solvents, for example xylene etc., are used.

If the compound of the formula (II) is in liquid form, no additional solvent is necessary; the compound of the formula (II) acts in this case both as starting material and also as solvent or diluent.

Preferably, the reaction is carried out without additional solvent or diluent.

The reaction temperature depends on the solvent possibly used, and on the starting materials and is between 70 and 200° C., preferably between 80 and 180° C., and particularly preferably between 100 and 160° C.

Then, water is added to the reaction mixture at 30 to 100° C. until all of the salt MX which may have precipitated out dissolves. This measure simultaneously saponifies any by-products possibly present in the form of orthoesters.

Furthermore, the addition of water leads to a phase separation, after which the low-boiling components in the form of alcohol R₁OH or R₂OH, for example methanol or ethanol, water, ROH and excess compound of the formula (III) or (II) are distilled off. The compound of the formula (IV) remains in the bottom and can, if desired, be purified by distillation.

The compound of the formula (IV) is, in most cases, for example in the case of benzyoxyacetaldehyde, very stable and is obtained in high purity. Owing to the stability, the compound of the formula (IV) can be stored over a relatively long period, so that the acetal cleavage to give the compound of the formula (I) need not be carried out immediately, but can be performed as required.

The dialkylacetal of the formula (IV), however, can also be fed, without any further purification, straight into the second step of the inventive process, the acetal cleavage.

The acetal cleavage is carried out by means of acid catalysis in the presence of transition metal catalysts, for example lanthanide catalysts.

Suitable catalysts for the acid catalysis are organic or inorganic acids, for instance sulfuric acid, p-toluenesulfonic acid, formic acid, acetic acid, oxalic acid, amidosulfonic acid etc.

Lanthanides which come into consideration are various compounds of cerium, lanthanum, ytterbium, samarium etc. These are, in particular, chlorides, sulfates and carboxylates.

Preferably, the acetal cleavage is carried out under acid catalysis. Particularly preferably, sulfuric acid is used for this.

The addition of water and the corresponding catalyst, preferably catalytic amounts of acid, and distilling off the eliminated alcohol, cleaves the dialkylacetal and converts it into the desired compound of the formula (I).

Water is added in this case in at least equimolar amount, or in molar excess, based on the acetal.

The end product is isolated by customary methods, for example extraction and subsequent purification by distillation.

By the means of the inventive process, the desired alkyl- or aryloxyacetaldehydes of the formula (I) are obtained in high yields and high purity in a simple manner, starting from readily accessible starting materials.

### EXAMPLE 1

a) Preparation of Benzyloxyacetaldehyde Dimethylacetal

648 g (6 mol) of benzyl alcohol were charged and 3 mol of sodium methoxide (30% in methanol) were added. 474 g of methanol were then distilled off, at approximately 135° C., 374 g (3 mol) of chloroacetaldehyde dimethylacetal were added in the course of 2 hours and allowed to react further for 2 hours at this temperature. NaCl precipitated out. After cooling to 70° C., 700 ml of water were added and the phases were separated. The organic phase (1011 g) was then worked up by distillation at 13 mbar via a column. 550 g of initial runnings contained excess benzyl alcohol and further low boilers, and at 125 to 128° C., 396 g of benzyloxyacetaldehyde dimethylacetal distilled over (67% yield, based on chloroacetaldehyde dimethylacetal used)

b) Acetal Cleavage to Give Benzyloxyacetaldehyde:

After the acetal (195 g, 1 mol) had been admixed with three times the amount of water, which had been set to pH 1 with sulfuric acid, methanol and water were distilled off at 70° C. and 300 mbar until no acetal was present any longer in the organic phase according to GC analysis. The benzyloxyacetaldehyde was then extracted twice with 300 ml of methyl t-butyl ether (MtBE). The organic phase was freed from MtBE on a Vapsilator and the residue was then distilled at 1 mbar and 75° C. The yield of benzyloxyacetaldehyde was 135 g (90%, based on acetal used).

1. A process for preparing alkyl- or aryloxyacetaldehydes of the formula 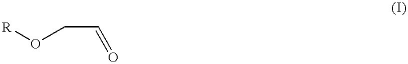
where R can be an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle, which comprises reacting a compound of the formula
R—OM (II)
where R is as defined above and M can be an alkali metal atom or an alkaline earth metal atom, with a compound of the formula 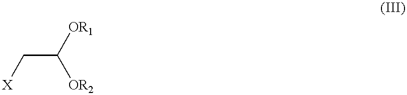
where R₁ and R₂ independently of one another are a C₁-C₆-alkyl radical or together are a C₂-C₆-alkylene radical and X is a halogen atom, to form the corresponding dialkylacetal of the formula 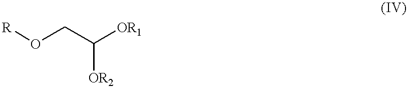
where R, R₁ and R₂ are as defined above, whereupon acetal cleavage is carried out to give the desired alkyl- or aryloxyacetaldehyde of the formula (I).

2. The process as claimed in claim 1, wherein, in the compound of the formula (I), R is a saturated or mono- or polyunsaturated, unbranched, branched or cyclic C₁-C₂₀-alkyl radical, a C₁-C₂₀-aryl radical or an alkaryl radical, in which case the radicals can be unsubstituted or mono- or polysubstituted by carboxylic esters or carboxylic amides, C₁-C₆-alkoxy, C₆-C₂₀-aryloxy, nitro or cyano.

3. The process as claimed in claim 1, wherein, in the compound of the formula (I), R is a saturated unbranched or branched C₂-C₈-alkyl radical or a benzyl radical, in which case the radicals can be unsubstituted or mono- or polysubstituted by carboxylic esters, C₁-C₆-alkoxy, C₆-C₂₀-aryloxy, nitro or cyano.

4. The process as claimed in claim 1, wherein, in the formula (II), M is Li, Na, K, Ca, Mg or Cs.

5. The process as claimed in claim 1, wherein, in the formula (III), R₁ and R₂ independently of one another are an unbranched or branched C₁-C₄-alkyl radical or, together, are a C₂-C₄-alkylene radical and X is Cl or Br.

6. The process as claimed in claim 1, wherein the compounds of the formula (II) and (III) are used in an equimolar amount or the compound of the formula (II) is used in a molar excess of 1.1 to 2 mol per mole of compound of the formula (III).

7. The process as claimed in claim 1, wherein the compound of the formula (II) acts as solvent.

8. The process as claimed in claim 1, wherein the compound of the formula (IV), after water has been added to dissolve any salt MX which may have precipitated out, M and X being defined as in formulae (II) and (III), is isolated from the reaction mixture and fed to the acetal cleavage.

9. The process as claimed in claim 1, wherein the acetal cleavage is carried out by acid catalysis using an organic or an inorganic acid.

10. The process as claimed in claim 1, wherein water in at lease equimolar amount, or in a molar excess, based on the acetal, is used for the acetal cleavage.
